(19) United States
(12) Patent Application Publication
(10) Pub. No.: US 2024/0207510 A1
(43) Pub. Date: Jun. 27, 2024

(54) DEVICE FOR ADMINISTERING LIQUIDS COMPRISING A DAMPING ELEMENT FOR ABSORBING EXTERNAL IMPACT FORCES

(71) Applicant: B. Braun Melsungen AG, Melsungen (DE)

(72) Inventors: Dirk Aljets, Kassel (DE); Klaus Angersbach, Morschen (DE); Christoph Erlen, Kassel (DE); Hans-Josef Gerlach, Marsberg (DE)

(21) Appl. No.: 17/918,937

(22) PCT Filed: Feb. 22, 2022
(86) PCT No.: PCT/EP2022/054344

§ 371 Date: Oct. 14, 2022

(30) Foreign Application Priority Data
Feb. 24, 2021 (DE) 10 2021 104 414.6

(51) Int. Cl. A61M 5/145; A61L 31/10; A61L 31/14

(52) U.S. Cl. CPC A61M 5/1456; A61L 31/10; A61L 31/14; A61M 2205/0238

(57) ABSTRACT

A device for administering a medical liquid, in particular a syringe pump. The device includes a main part into which a hollow body containing the liquid to be administered is receivable. The device includes a drive for acting on the hollow body such that the liquid is conveyed. The drive includes a drive head projecting from the main part. The device also includes a damping element disposed on an outside of the drive head for absorbing external impact forces acting on the drive head.

### CROSS-REFERENCE TO RELATED APPLICATIONS

This application is the United States national stage entry of International Application No. PCT/EP2022/054344, filed Feb. 22, 2022, and claims priority to German Application No. 10 2021 104 414.6, filed Feb. 24, 2021. The contents of International Application No. PCT/EP2022/054344 and German Application No. 10 2021 104 414.6 are incorporated by reference herein in their entireties.

### FIELD

The present disclosure relates to a device for administering a medical liquid. In particular, the disclosure relates to an infusion pump, preferably a syringe pump.

### BACKGROUND

Infusion pumps are substantially distinguished between syringe pumps and peristaltic pumps. In the case of syringe pumps, a syringe filled with the liquid to be administered is moved by a drive so that a syringe plunger of the syringe is moved at a controlled feed velocity to convey the liquid out of the syringe. In the case of peristaltic pumps, a tube filled with the liquid to be administered is moved by a drive, for example by means of peristalsis, to convey the liquid out of the tube.

Such a syringe pump is known from WO 2019/213 496 A, for example. The syringe pump includes a housing in which a syringe can be received and a drive laterally projecting from the housing.

Previously known syringe pumps already have impact protection to protect their drive. Due to an increasing size and/or an increasing weight of newly developed syringe pumps, the damping of the previous impact protection is not sufficient so that the drive, particularly a drive head of the drive, is damaged when it falls onto the drive head.

It is further known to arrange a protective bracket around endangered areas of the syringe pump, which, however, entails the drawback that the dimensions of the syringe pump increase, the syringe pump with a protective bracket is visually less attractive and is more complicated to clean.

It is another known solution for protecting the syringe pump that the components of the syringe pump as such, in particular the components occurring in the chain of force in the event of a fall, are designed to be very robust so that they are not damaged, if the syringe pump falls onto said components. Such robust design renders the syringe pump as a whole more expensive, heavier and larger, however, which should be avoided.

Therefore, it is the object of the present disclosure to provide a device for administering a medical liquid, in particular a syringe pump, which has a compact design and, at the same time, can ensure its functionality even in the event of a fall. In addition, the device is intended to be easy to clean and to be manufactured at low cost.

In particular, the device for administering a medical liquid is in the form of a syringe pump. A syringe pump is understood to be a dosing pump by which the medical liquid contained in a syringe can be administered continuously to a patient by axial movement of a plunger/piston of the syringe. Syringe pumps are frequently designated with their trade name as perfusors.

The device has a main part. In the main part a hollow body can be at least partly received. The hollow body that can be received in the main part of the device contains the liquid to be administered. In the case of a syringe pump, the hollow body is designed as a cylindrical hollow body of a syringe.

The device includes a drive. The drive is suitable and designed for acting on the hollow body such that the liquid is conveyed. In the case of a syringe pump, the drive is particularly suitable for axially moving a plunger of the syringe so that the contained liquid is pressed out of the syringe. The drive includes a drive head projecting from the main part of the device.

According to the present disclosure, the device includes a damping element. The damping element is disposed on an outside of the drive head for absorbing external impact forces acting onto the drive head, in particular for absorbing kinetic energy in the event of a drop/fall of the device onto the drive head. Hence, this means that the damping element is arranged on the exterior of the drive head so that, in the event of a drop/fall of the device, at first the damping element contacts the ground before the external impact forces act on the drive head itself. In this way, the impact energy acting on the drive head can be buffered/reduced.

This offers the advantage that the drive head which is particularly vulnerable due to its arrangement projecting from the main part and at the same time is essential for the drive and, thus, for conveying the device, is additionally protected.

According to a preferred embodiment, the drive head can be arranged in the axial direction relative to the hollow body (or a receiving region of the main part which is suitable for receiving the hollow body). That is to say, the drive head projects, in a position of use of the device, laterally from the main part.

For example, the drive may be designed as a linear drive. In particular, the linear drive may include a drive motor and a spindle coupled to the drive motor. Such linear drive changes its axial position and projects far from the main part of the device in particular when the syringe is completely filled so that it should be specifically protected.

According to a preferred embodiment, the damping element may be provided on an end face of the drive head projecting from the main part so as to absorb impact forces acting in the axial direction on the drive head. This offers the advantage that the impact forces can be absorbed particularly well in the event of a drop/fall along the axial direction, i.e., in the event of vertical fall, onto the ground.

According to another preferred embodiment, the damping element can envelop the drive head projecting from the main part at least partly to absorb impact forces in the radial and axial directions onto the drive head. The enveloping arrangement also helps to absorb the drop forces occurring when the device hits the ground in a tilted position. In this way, the drive head is protected at all times.

According to a preferred embodiment, the damping element can be made of a polyurethane-based gel, specifically of technogel. Such gel offers particularly good shock-absorbing characteristics and, consequently, is suited for use as a shock absorber.

According to the preferred embodiment, the damping element may be designed such that the gel deforms for absorbing kinetic energy in case the device falls onto the drive head. The deformation of the gel may be an elastic deformation, which offers the advantage that the device can be continued to be used after a fall without the gel having to be replaced. The deformation of the gel may also be a plastic deformation, which offers the advantage that higher impact forces can be absorbed. The deformation of the gel may also be partly elastic and partly plastic so that the advantages are combined, and the gel can be continued to be used in the case of minor impacts, for example, and must be replaced in the case of major impacts.

According to the preferred embodiment, the gel may have a honeycomb structure, the honeycombs preferably projecting in a direction away from the drive head. A honeycomb structure is a suitable option for allowing the gel to deform.

According to another preferred embodiment, the damping element may be made of silicone. Silicone dampers have proven to be both particularly effective for shock absorbing and capable of being manufactured at low cost.

According to the other preferred embodiment, the damping element may be designed such that, for absorbing kinetic energy in the event of a fall of the device onto the drive head, it alters the air pressure of air enclosed in the silicone. Hence, this means that the kinetic energy can be converted at least partly into pressure energy.

According to the other preferred embodiment, the damping element made of silicone may have a honeycomb structure, the honeycombs preferably projecting in a direction away from the drive head. A honeycomb structure is a suitable option for allowing the kinetic energy to be converted into pressure energy of the air which is enclosed in the individual honeycombs.

According to a preferred embodiment, the device can include a cap and/or a cover which covers the exterior of the damping element and/or secures the damping element to the drive head. For example, the cap can be made of plastic as this is particularly easy to clean. The cap under which the damping element is located can help ensure easy securing of the damping element to the drive head. Preferably, the damping element can be arranged detachably on the drive head, for example by the cap. In this way, the damping element can be replaced if needed.

According to the preferred embodiment, the cap can be arranged on the drive head preferably by an elastic form closure, such as a snap connection. This means that the cap is arranged without any connecting element, in particular directly, on the drive head so that no connecting elements are projecting on which impurities might collect. In one embodiment, the elastic form closure can be designed by a recess located on the outside of the drive head in which a projection located on an inside of the cap engages. Alternatively, the elastic form closure can be designed by a recess in the inside of the cap in which a projection located on the outside of the device engages.

According to a preferred embodiment, the damping element can be dimensioned depending on a drop height to be maximally expected during normal use, specifically 1.80 m, and/or on a weight of the device. In this way, the device can be ensured not to be damaged in the event of fall depending on its use.

In other words, the present disclosure relates to a device for administering a medical liquid, in particular a syringe pump, in which the drive head is equipped with a damping element for protection. This damping element reduces the forces acting on the device and the components thereof if the device falls onto hard ground, to an uncritical extent. The impact protection in the drive head is selected such that the potential energy released in the event of a fall is converted to the deformation of the damping element and, resp., to the conversion into pressure energy of air enclosed in the damping element. Thus, the drive head can continue to convey even in the event of a fall directly onto the drive head, and internal components of the device are not damaged. Preferably, the impact protection of the syringe pump is formed by a cap/impact protection cap and a damping element disposed under the cap and made of a technogel, i.e., a polyurethane-based gel, or a honeycomb structure disposed under the cap and made of silicone. The impact protection dampens a drop/fall such that the drive is not damaged when it falls onto the drive head. Summing up, the syringe pump has an additional damping element located under a plastic cap which mitigates a shock so that, in the event of a fall onto the drive head, the drive remains functional even if the weight of the device increases and the drive is subjected to higher forces when it falls.

### BRIEF DESCRIPTION OF THE DRAWING FIGURES

FIGS. 1 and 2 are perspective representations of a device according to the present disclosure comprising a damping element.

### DETAILED DESCRIPTION

In the following, a preferred embodiment of the present disclosure will be described on the basis of the associated Figures.

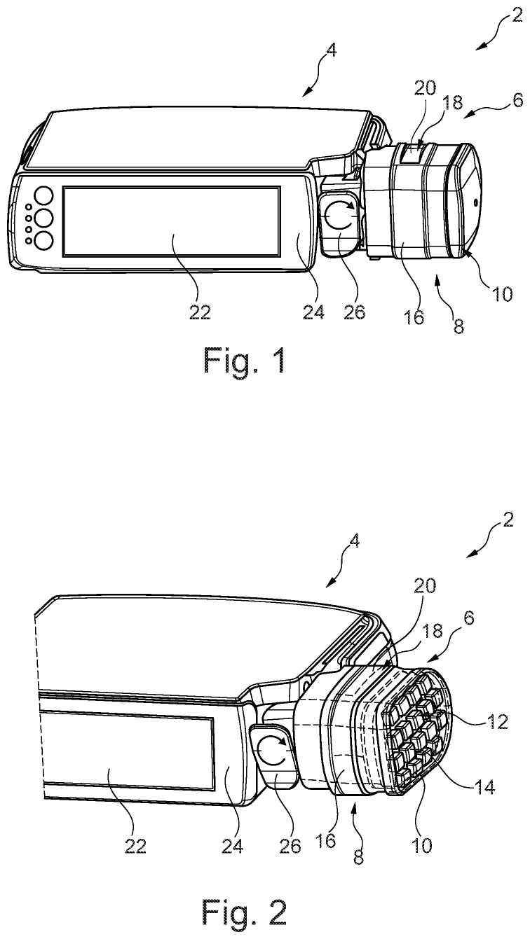

FIGS. 1 and 2 illustrate a device 2 for administering a medical liquid. The device 2 can be designed particularly as a syringe pump. The device 2 has a main part 4. In the main part 4, a hollow body (not shown) can be at least partly received. The hollow body receivable in the main part 4 of the device 2 contains the liquid to be administered. In the case of a syringe pump, the hollow body is designed as a cylindrical hollow body of a syringe.

The device 2 comprises a drive 6. The drive 6 is suited and designed to act on the hollow body such that the liquid is conveyed. In the case of a syringe pump, the drive 6 is particularly suitable for axially moving a plunger of the syringe so that the contained liquid is pressed out of the syringe. The drive 6 includes a drive head 8 projecting from the main part 4 of the device 2. The drive 6 is preferably designed as a linear drive. The drive head 8 may be arranged in an axial direction relative to the hollow body. That is, in a position of use of the device 2, the drive head 8 projects laterally from the main part 4.

The device 2 comprises a damping element 10. The damping element 10 is disposed on an outside of the drive head 8 for absorbing external impact forces acting on the drive head 8. In particular, the damping element 10 serves for absorbing kinetic energy, if the device 2 drops/falls onto the drive head 8. Preferably, the damping element 10 can be arranged detachably on the drive head 8.

According to a preferred embodiment, the damping element 10 can be dimensioned depending on a maximum height of fall to be expected during normal use, specifically 1.80 m, and/or on a weight of the device.

The damping element 10 preferably can be provided on an end face of the drive head 8 projecting from the main part 4 so as to absorb impact forces acting in an axial direction on the drive head 8. As an alternative, the damping element 10 can at least partly envelop the drive head 8 projecting from the main part to absorb impact forces acting in radial and axial directions on the drive head.

The damping element 10 can be made specifically of a polyurethane-based gel, specifically of a technogel. Preferably, the damping element 10 can be designed such that, for absorbing kinetic energy if the device 2 falls onto the drive head 8, the gel deforms plastically and/or elastically.

As an alternative, the damping element 10 can be made of silicone. Preferably, the damping element 10 can be designed such that it alters the air pressure of air enclosed in the silicone for absorbing kinetic energy if the device 2 falls onto the drive head 8. Hence, this means that the kinetic energy can be converted at least partly into pressure energy.

It is evident particularly from FIG. 2 that the damping element 10 may have a honeycomb structure. This means that the damping element 10 has honeycomb projections 12 which are separated from each other by gaps 14 between the individual honeycomb projections 12. Preferably, the honeycomb projections 12 project in a direction, such as an axial direction, away from the drive head.

In addition, the device 2 may comprise a cap (/cover) 16. The cap 16 is transparent in FIG. 2 so that the components disposed under the cap 16, in particular the damping element 10, are visible.

The cap 16 can cover the exterior of the damping element 10 and/or can secure the damping element 10 to the device 2, in particular to the drive head 8. Preferably, the damping element 10 is disposed completely under the cap 16. In this way, in particular impurities are prevented from settling on the damping element 10, specifically on the honeycomb structure. The cap 16 may be a plastic cap.

The cap 16 can secure the damping element 10 detachably to the drive head 8. Preferably, the cap 16 can be arranged on the drive head 8 by an elastic form closure, such as a snap connection. That is, the cap 16 can be arranged on the drive head 8 in particular directly without any connecting element. In the illustrated embodiment, the elastic form closure can be designed by a recess 18 located on the outside of the device 2 (of the drive head 8) in which a projection 20 located on an inside of the cap 16 engages. Although not shown, the elastic form closure can alternatively be designed by a recess in the inside of the cap 16 in which a projection on the outside of the device 2 (of the drive head 8) engages.

Additionally, the device 2 comprises a display unit 22 for graphical representation of parameters of the device 2 relevant to operation. The display unit 22 is integrated in a cover 24 that can be opened by actuating a lever 26 to load the hollow body into the main part 4 of the device 2 and to remove the hollow body from the main part 4 of the device.

1. A device for administering a medical liquid, the device comprising:
a main part into which a hollow body containing the medical liquid is at least partly receivable;
a drive configured for acting on the hollow body such that the medical liquid is conveyed, the drive comprising a drive head projecting from the main body; and
a damping element is disposed on an outside of the drive head for absorbing external impact force acting on the drive head.

2. The device according to claim 1, wherein the damping element is provided on an end side of the drive head to absorb impact force acting in an axial direction on the drive head.

3. The device according to claim 1, wherein the damping element at least partly envelops the drive head to absorb impact force acting in radial and axial directions on the drive head.

4. The device according to claim 1, wherein the damping element is made of a gel that is polyurethane-based.

5. The device according to claim 4, wherein the damping element is designed such that the gel deforms for absorbing kinetic energy.

6. The device according to claim 1, wherein the damping element is made of silicone.

7. The device according to claim 6, wherein the damping element is designed such that the damping element alters a pressure of air enclosed in the damping element to absorb kinetic energy.

8. The device according to claim 4, wherein the damping element has a honeycomb structure.

9. The device according to claim 1, further comprising a cap (that covers the damping element and/or secures the damping element to the drive head, the cap being arranged on the drive head.

10. The device according to claim 1, wherein the damping element is dimensioned depending on a maximum fall height to be expected during normal use and/or on a weight of the device.

11. The device according to claim 1, wherein the device is a syringe pump.

12. The device according to claim 1, wherein the damping element is disposed on an exterior of the drive head for absorbing kinetic energy.

13. The device according to claim 4, wherein the gel is a technogel.

14. The device according to claim 8, wherein the honeycomb structure comprises honeycombs that project in a direction away from the drive head.

15. The device according to claim 9, wherein the cap is made of plastic.

16. The device according to claim 9, wherein the cap detachably secures the damping element to the drive head.

17. The device according to claim 9, wherein the cap is arranged on the drive head by an elastic form closure.

18. The device according to claim 10, wherein the maximum fall height to be expected during normal use is 1.80 m.
